(19) United States
(12) Patent Application Publication
(10) Pub. No.: US 2023/0009050 A1
(43) Pub. Date: Jan. 12, 2023

(54) ERGONOMIC GAUZE PAD HOLDER FOR POSTSURGICAL INTRAORAL USE

(71) Applicant: TALA SURGICAL INC., Hatillo, PR (US)

(72) Inventor: Cesar R. Talavera-Peraza, Hatillo, PR (US)

(73) Assignee: TALA SURGICAL INC., Hatillo, PR (US)

(21) Appl. No.: 17/370,459

(22) Filed: Jul. 8, 2021

(51) Int. Cl. A61F 13/38

(52) U.S. Cl. CPC A61F 13/38

(57) ABSTRACT

An ergonomic gauze pad holder, useful in holding an in site after an intraoral surgical procedure is disclosed. The use of the gauze pad holder foments and promotes a hygienic and safe clot formation in an intraoral extraction site after tooth extraction. The design of the holder in terms of its use includes an external section and an internal section that may be connected in different positions, relative to the position of the intraoral site wherein the gauze pad is intended to be used. The external section includes a handle and connecting section that allows the user to control the holder from the exterior of his mouth. The internal section, intended to be used inside the patient mouth, has two internal receiving channels wherein two extended inserting sections are inserted and secured creating an internal cavity wherein a gauze pad may be inserted secured, and assembled in different configurations. The particular configuration of the gauze pad depends on the particular position inside the intraoral site wherein the gauze pad is intended to be used. The external surface of the internal section is smooth, and it is designed ergonomically to be comfortably housed in the patient mouth. The whole design of the ergonomic gauze pad holder herein disclosed is directed to reduce the risks of complications after tooth extractions, reduce the healing time, and increase the safety of the patient during the post-surgical healing process.

### TECHNICAL FIELD OF THE INVENTION

The invention herein disclosed relates to gauze pad holders. More specifically, it is directed to ergonomic gauze pad holders, useful in firmly securing and holding a sanitized gauze pad at a particular area of the intraoral cavity of a patient after a surgical procedure has been performed.

### BACKGROUND OF THE INVENTION

After the extraction of a tooth, the formation of a blood clot inside of the socket wherein the extracted tooth used to be is an initial and essential step in the post-extraction healing process of the extraction site. Such blood clot is formed inside the socket and on the bone exposed at the site of the tooth extraction. This blood clot provides a protective layer over the underlying bone and nerve endings in the empty tooth socket. Said clot also provides the foundation for the growth of new bone and the development of soft new tissue formed over the clot.

In some instances, and due to different factors, said blood clot is not always properly formed at the extraction site. It may, for example, be dislodged or dissolved previous to the healing of the wound. Such lack of clot formation and thus, lack of wound healing, as the consequence of exposing the underlying bone and nerves related to the socket to external environmental conditions such as air, food, fluids, bacteria, and any other potentially opportunistic microorganisms; that results in intense pain in the socket as well as in the nerves radiating to the side of the patient's face. Such a condition may produce excess bleeding and, more importantly, it causes Alveolar osteitis, most commonly known as “dry socket”. Therefore, promoting a safe healing process requires prophylactic measures directed to induce a healthier, and hygienic environment in the extraction site to promote proper blood clot formation.

Conventionally, to control the gums from bleeding excessively after the extraction procedure has been completed and more particularly to properly promote the blood clot formation in the right position within the socket, the common practice is to place a gauze pad on the socket or extraction site and request the patient to bite on it for 30 minutes so that pressure is applied and maintained in the surgical area. In this manner, the socket area is maintained dry and covered, thus it is protected from contact with external materials, microorganisms, and other sorts of pathogens. Nonetheless, such a conventional procedure has a series of disadvantages. For example, said gauze pad is usually inserted into the postsurgical intraoral area by healthcare personnel, personal assistants, or by the patient himself, generally by introducing their hands into the patient's mouth. Such a conventional process represents a poor hygienic practice that exposes the surgical area to potential pathogens such as bacteria and/or other microorganisms, increasing the chances of opportunistic infections and other postsurgical complications. Indeed, presently, infections involving antibiotic-resistant bacteria are a real challenge to treat, thus new preventive or prophylactic measures with the aim of avoiding exposure to such bacteria are an essential part of any proper postsurgical healthcare procedure.

Another potentially dangerous situation is that when the gauze pad is not secured by any means inside the oral cavity of a patient, who is generally under some kind of sedation, said gauze pad may be displaced from the surgical area to the patient's pharynx, causing an obstruction of the patient's airways, thus representing asphyxiation or a choking hazard. In a similar manner, the lack of the patient's visual contact with the exact surgical area increases the chance that the patient may not be able to properly position the gauze pad in the proper place by himself, thus no pressure is maintained in the postsurgical area and no proper formation of the blood clot is achieved. Similarly, the removal of the already used gauze pad inside the postsurgical site is an unpleasant and uncomfortable task, since said gauze pad is soaked with blood and saliva, thus there is a probability that such removal also displaces the initial blood clot that is in the initial process of formation. Moreover, to be effective in the postsurgical healing process, the gauze pad should be folded properly, preferably in a square shape by folding it in half twice, previous to be inserted in the postsurgical area or extraction site. Furthermore, if a holder is used to provide holding support to said gauze, the design of said holder requires an ergonomic shape, able to be accommodated to the particular shape of the interior of the patient's mouth to reduce the patient's uncomfortable sensation and further protect the integrity of the gums and other teeth.

Therefore, there is a need to provide an ergonomic, suitable, hygienic, and sanitary gauze pad holder that firmly supports and holds a gauze properly assembled on it and that allows the control of the gauze pad over the particular and specific postsurgical intraoral area in order to eliminate or reduce postsurgical oral complications, and simultaneously is comfortable to use, maintain and discard.

### Objectives of the Invention

It is an objective of the invention to provide a gauze pad holder suitable to firmly hold a disinfected and sanitized gauze pad in a postsurgical intraoral area under controlled ergonomic, safe, hygienic, secure, and sanitary conditions. Another objective of the invention is to provide an ergonomic gauze pad holder suitable to receive and firmly secure a gauze pad useful as a prophylactic measure directed to avoid exposure of antibiotic-resistant bacteria and other pathogens or microorganisms in a postsurgical intraoral site. A further objective of the invention is to provide a gauze pad holder having an ergonomic design with smooth external surfaces, that is easy to be accommodated in the interior of a human mouth while supporting and securing a hygienic gauze pad assembled conveniently, and without producing discomfort in the patient gums, while simultaneously, allowing a proper formation of a blood clot in the intraoral postsurgical site. Yet another aim of the invention is to provide an ergonomic gauze pad holder as a tool for improving the conditions for the healing process of a postsurgical intraoral site by reducing or minimizing the chances of a blood clot to be dislodged and thus avoiding medical conditions, such as Alveolar osteitis, most commonly known as “dry socket”.

Even a further objective of the instant invention is to provide an ergonomic gauze pad holder that eliminates the need to insert a hand inside the patient's mouth to place a gauze pad into the postsurgical intraoral site, while positioning said gauze pad in a proper position for the required time needed to facilitate the formation of the blood clot. Yet another aim of the invention is to provide an ergonomic gauze pad holder that provides control to the patient in maintaining a gauze pad firmly, properly, and safely, in the right position in the postsurgical intraoral site, thus avoiding the blood clot displacement from said site, in order to increase the effectiveness of the gauze pad in stopping the gums bleeding and simultaneously avoiding the potential choking of the patient caused by the gauze pad. In yet another objective, the instant invention provides an ergonomic gauze pad holder that allows the patient to have external control of the gauze pad, without the need of inserting the fingers in the patient's intraoral cavity with the intention to re-accommodate said pad, thus drastically reducing potential risks of contamination. Another goal of the instant invention is to provide a gauze pad holder that facilitates the use and maintenance of a gauze pad already assembled in a secure and proper stable folding and shape manner, during the process that said gauze pad is used in the extraction site, to ensure proper or ideal contact of the said pad with the postsurgical site. Yet another aim of the instant invention is to provide a gauze pad holder having an ergonomic structure that is comfortably positioned over the postsurgical site inside the intraoral cavity and that allows a gauze pad to be assembled and secured in different positions, depending on the particular configuration of the intraoral site requiring the use of said gauze pad and without negatively affecting the patient's gums.

### BRIEF DESCRIPTION OF THE DRAWINGS

The foregoing and additional features and characteristics of the present invention will become more apparent from the following detailed description considered in reference to the accompanying drawings, wherein:

FIG. 1 shows a perspective view of the general concept of the gauze pad holder according to the invention.

FIGS. 2 and 3 illustrate perspective views of one of the embodiments of the gauze pad holder according to the invention in the open position, wherein the connecting section is connected to the back side of the first holding section.

FIGS. 4 and 5 are perspective views of the embodiment of the gauze pad holder illustrated in FIGS. 2 and 3 in a closed configuration and showing the internal cavity wherein the gauze pad may be firmly held and secured.

FIGS. 6 and 7 illustrate perspective views of another of the embodiments of the gauze pad holder according to the invention in the open position, wherein the connecting section is connected to the back side of the supporting section, more particularly, between that first holding section and the second holding section.

FIGS. 8 and 9 are perspective views of the embodiment of the gauze pad holder illustrated in FIGS. 6 and 7, in a closed configuration and showing the internal cavity wherein the gauze pad may be firmly held and secured.

FIG. 10 is a front view of the close configuration of the gauze pad holder without the gauze pad, showing the first inserting section and the second inserting section already inserted into the first receiving internal channel and the second receiving internal channel, respectively, thus providing the internal cavity suitable to firmly hold and secure a gauze pad.

FIG. 11 is a perspective view of the embodiment of the gauze pad holder illustrated in FIGS. 2, 3, 4, and 5, having a gauze pad already assembled in a rolled-up configuration and firmly secured in its internal cavity.

FIG. 12 is a perspective view of the embodiment of the gauze pad holder illustrated in FIGS. 2, 3, 4, and 5, having a gauze pad already assembled in a folded-up configuration and firmly secured in its internal cavity.

FIG. 13 is a perspective view of the embodiment of a gauze pad holder according to the invention illustrated in FIGS. 6, 7, 8, and 9 having a gauze pad already assembled in a rolled-up configuration and firmly secured in its internal cavity.

FIG. 14 is a perspective view of the embodiment illustrated in FIGS. 6, 7, 8, and 9, having a gauze pad already assembled in a folded-up configuration and secured in its internal cavity.

FIGS. 15, 16, 17, and 18 show perspective views illustrating the preferred manner to handle different embodiments of the gauze pad holder according to the instant invention.

FIG. 19 shows a perspective view of the postsurgical use of the embodiment of the gauze pad holder illustrated in FIG. 11, used in the socket located at the third molar tooth site after the intraoral surgical procedure has been performed.

FIG. 20 shows a perspective view of the postsurgical use of the embodiment of the gauze pad holder illustrated in FIG. 13 as used in the socket located at the third molar tooth site after the intraoral surgical procedure has been performed.

FIG. 21 shows a perspective view of the postsurgical use of the embodiment of the gauze pad holder illustrated in FIG. 12, used in the socket located at the second molar tooth site after the intraoral surgical procedure has been performed.

FIG. 22 shows a perspective view of the postsurgical use of the embodiment of the gauze pad holder illustrated in FIG. 14, used in the socket positioned at the first molar site after the intraoral surgical procedure has been performed.

FIG. 23 shows a perspective view of the postsurgical use of the embodiment of the gauze pad holder illustrated in FIG. 12, used in the socket at the canine tooth site after the intraoral surgical procedure has been performed.

FIG. 24 shows a perspective view of the postsurgical use of the embodiment of the gauze pad holder illustrated in FIG. 14, used in the socket located at the central incisor tooth site after the intraoral surgical procedure has been performed.

FIG. 25 shows a patient with his mouth closed and using any of the gauze pad holder embodiments herein disclosed after an intraoral surgical procedure has been performed on any molar or premolar tooth site at the left side of his mouth.

FIG. 26 shows a patient with his mouth closed and using any of the gauze pad holder embodiments herein disclosed after an intraoral surgical procedure has been performed on any of his front teeth.

FIG. 27 shows a patient with his mouth closed and using any of the gauze pad holder embodiments herein disclosed after an intraoral surgical procedure has been performed on any molar or premolar tooth site at the right side of his mouth.

FIG. 28 shows a sanitary and disposable kit containing a predetermined number of individually wrapped units of the gauze pad holder illustrated in FIG. 11, wherein the enclosure of said kit has been cut off to show its details.

FIG. 29 shows a view of the embodiment of the gauze pad holder illustrated in FIG. 11, already sanitized, and individually wrapped in a sanitary and disposable bag, and ready to be packed in the kit illustrated in FIG. 28.

FIG. 30 shows a sanitary and disposable kit containing a predetermined number of individually wrapped units of the gauze pad holder illustrated in FIG. 12, wherein the enclosure of said kit has been cut off in order to show its details.

FIG. 31 shows a view of the embodiment of the gauze pad holder illustrated in FIG. 12, already sanitized, and individually wrapped in a sanitary and disposable bag ready to be packed in the kit illustrated in FIG. 30.

FIG. 32 shows a sanitary and disposable kit containing a predetermined number of individually wrapped units of the gauze pad holder illustrated in FIG. 13, wherein the enclosure of said kit has been cut off to show its details.

FIG. 33 shows a view of the embodiment of the gauze pad holder illustrated in FIG. 13, already sanitized, and individually wrapped in a sanitary and disposable bag ready to be packed in the kit illustrated in FIG. 32.

FIG. 34 shows a sanitary and disposable kit containing a predetermined number of individually wrapped units of the gauze pad holder illustrated in FIG. 14, wherein the enclosure of said kit has been cut off to show its details.

FIG. 35 shows a view of the embodiment of the gauze pad holder illustrated in FIG. 14, already sanitized, and individually wrapped in a sanitary and disposable bag ready to be packed in the kit illustrated in FIG. 34.

### DETAILED DESCRIPTION OF THE EMBODIMENTS

The following detailed description illustrates the instant invention and a variety of embodiments by way of example and is not limited to the particular limitations presented herein as principles of the invention. This description is directed to enable one skilled in the art to make and use the invention by describing embodiments, adaptations, variations, and alternatives of the invention. Any potential variations of the limitations herein described are within the scope of the invention. The drawings are not necessarily to scale; some features may be exaggerated or minimized to show details of particular components of the invention. It is also to be understood that the terminology used herein is to describe particular embodiments of the invention only and is not intended to limit the scope of the invention in any manner.

It must be noted that, as used in this specification and the appended claims, the singular forms “a”, “an” and “the” include plural referents unless the context dictates otherwise. Therefore, specific structural and functional details disclosed herein are not to be interpreted as limiting, but merely as a representative basis for the claims and/or as a representative basis for teaching one skilled in the art to variously employ the present invention.

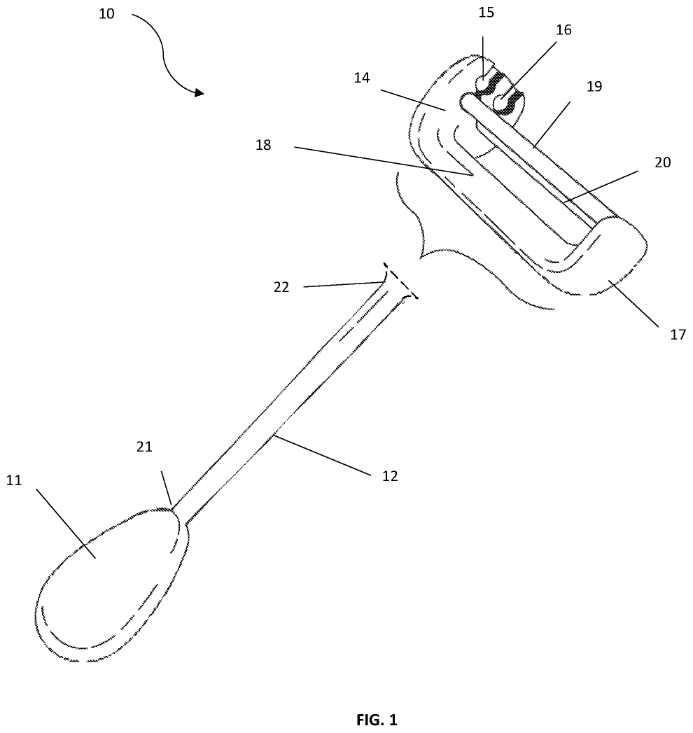

In general terms, the instant invention is directed to an ergonomic gauze pad holder 10, useful in firmly holding and securing a gauze pad, which is intended to be pressed or bitten in a post-surgical precise location inside the mouth, just after a surgical dental procedure has been performed. The instant invention comprises different embodiments of the ergonomic gauze pad holder 10, capable to be adapted to the particular intraoral postsurgical location. All said embodiments according to the invention shares the same structural sections, thus all of them comprise the following common sections: a handle section 11, a connecting section 12, a first holding section 14, a second holding section 17, a supporting section 18, a first inserting section 19, and a second inserting section 20 as main sections; which are all structurally identical in all embodiments of said ergonomic gauze pad holder 10. All mentioned main sections are physically integrated in the single functional physical structure that provides the ergonomic gauze pad holder 10. The main difference between the different embodiments according to the invention, exclusively depends on how the connecting section 12 is positioned or connected in the structure of the gauze pad holder 10, as discussed below, and suggested in FIG. 1.

Regarding the drawings, in FIG. 1, it is illustrated a perspective view of the general concept of the gauze pad holder 10 according to the invention, and in close configuration. It shows handle 11, preferably comprising an oval-shaped body with a concave center, which is surrounded by rounded edges. It also shows connecting section 12, which comprises a main elongate and cylindrical body having a first end 21 and a second end 22, wherein said first end 21 is connected to said handle section 11. FIG. 1 also shows first holding section 14, which comprises a main body having a first internal receiving channel 15, and a second internal receiving channel 16. Furthermore, it also shows supporting section 18, second holding section 17, first inserting section 19, and second inserting section 20. Said gauze pad holder 10 integrates all said main sections as a single functional unit, that is intended to be used intraorally, and has ergonomic features that conveniently adapt to the internal shape of the human mouth, particularly considering the curvature of the interior of the oral cavity and the particular position of the socket originated after an intraoral surgical procedure has been performed. Individual and particular embodiments of the gauze pad holder 10 comprising the mentioned main sections as a single unit are described below.

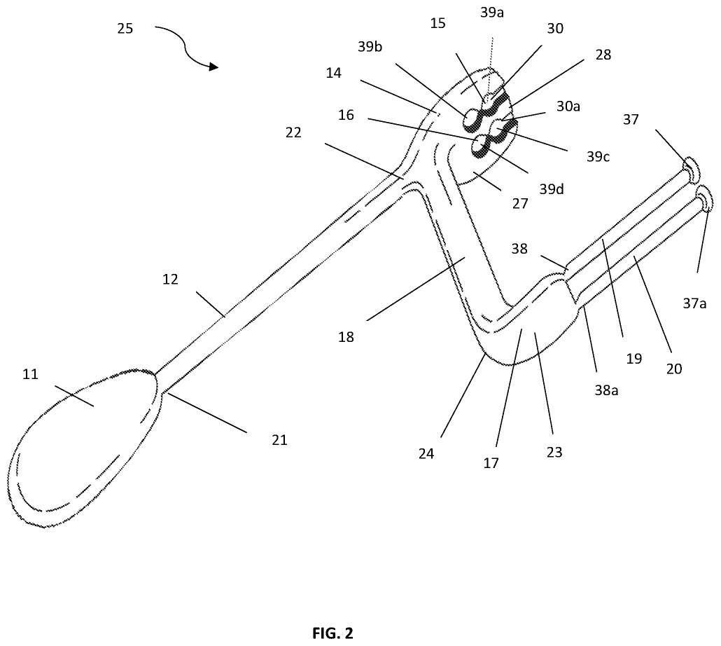

As suggested in FIG. 1, the second end 22 of the cylindrical body of the connecting section 12 may be connected (1) directly and in an aligned position to the first holding section 14; (2) directly to the supporting section 18; or (3) directly and in aligned position to the second holding section 17. For instance, in FIGS. 2, 3, 4, and 5, it is shown preferred embodiment 25, which according to the invention and as indicated previously, comprises handle section 11, connecting section 12 having its first end 21 connected to said handle section 11, first holding section 14 which comprises first internal receiving channel 15 and second internal receiving channel 16, second holding section 17, supporting section 18, first inserting section 19 and second inserting section 20. As illustrated in FIGS. 2, 3, 4, and 5, in embodiment 25, the second end 22 of connecting section 12 is connected to the back side of and aligned to the first holding unit 14.

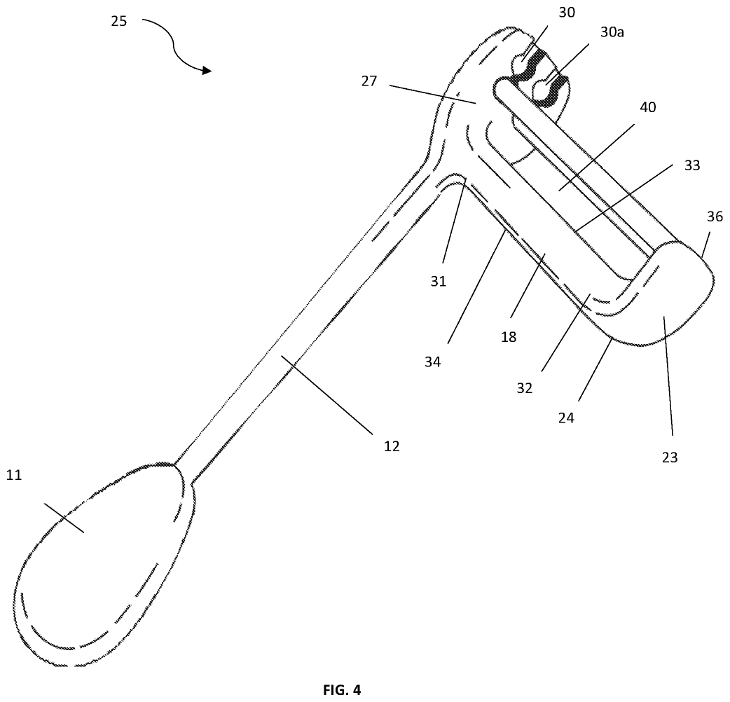

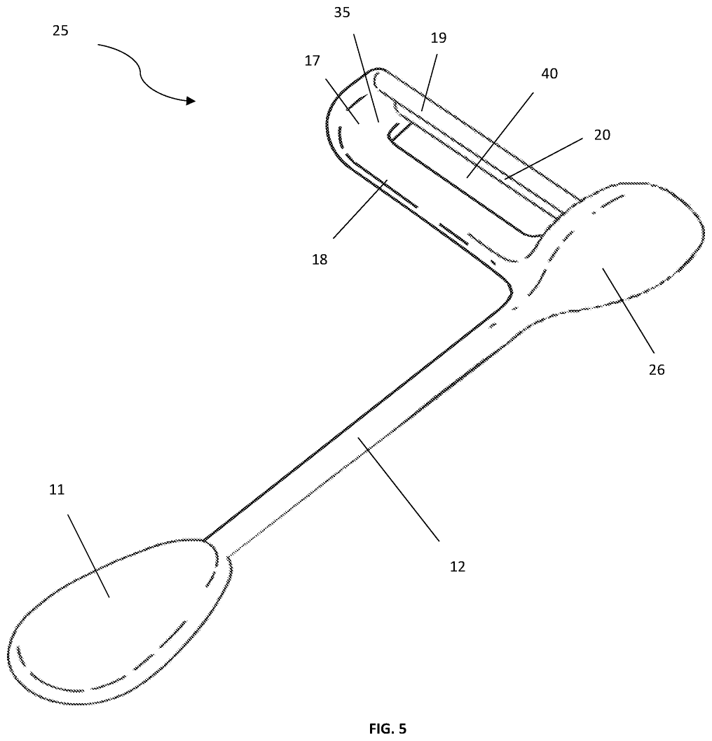

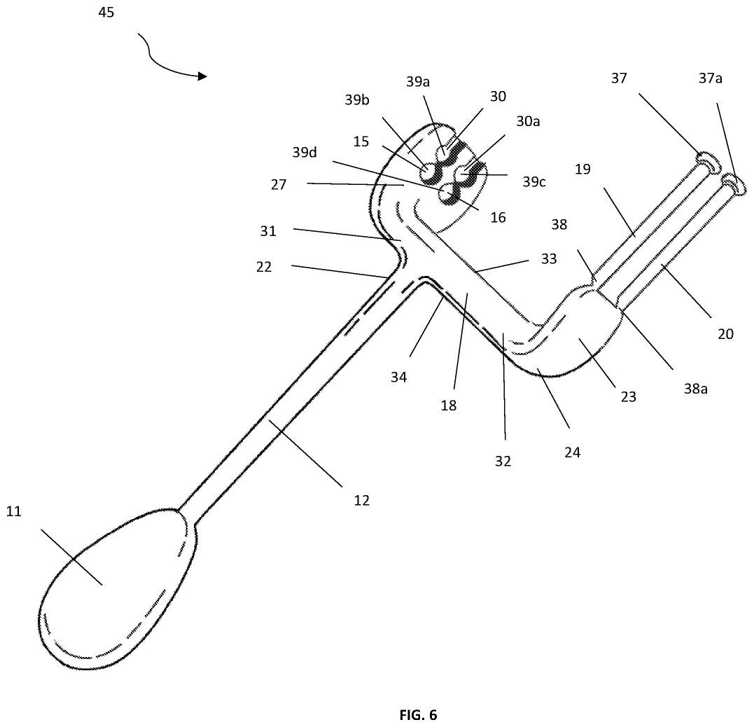

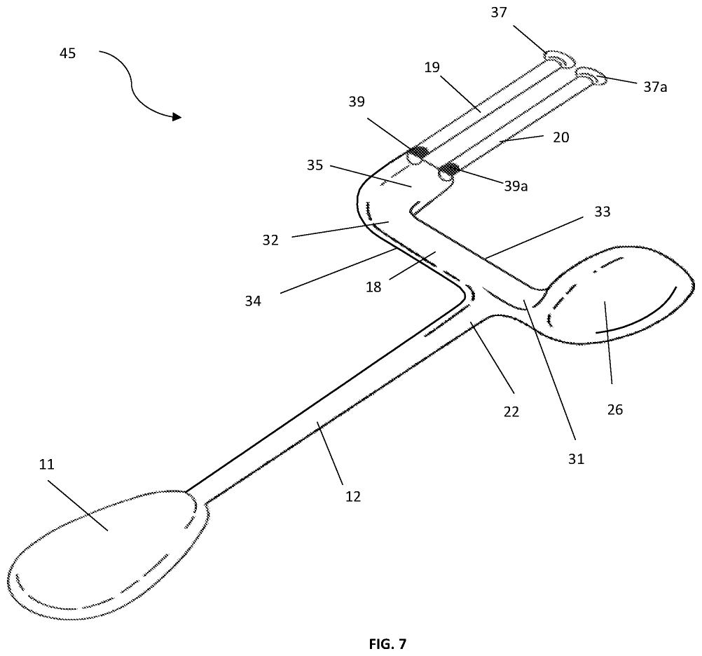

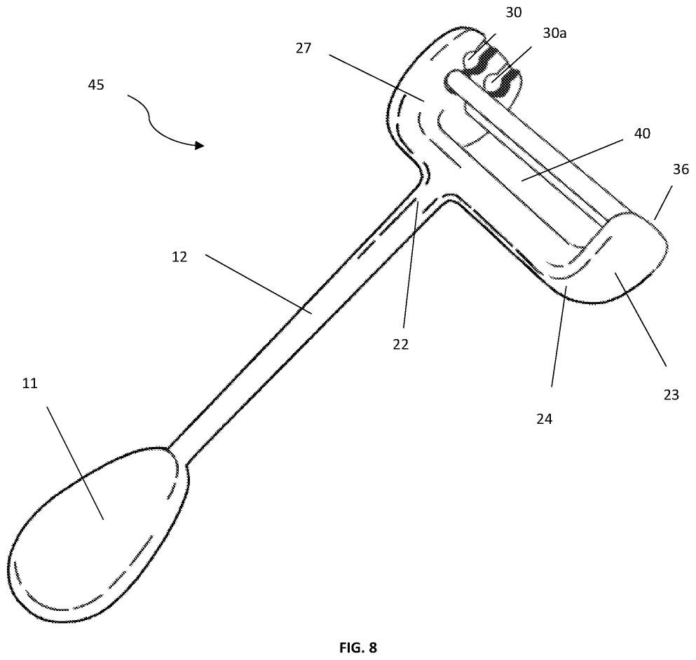

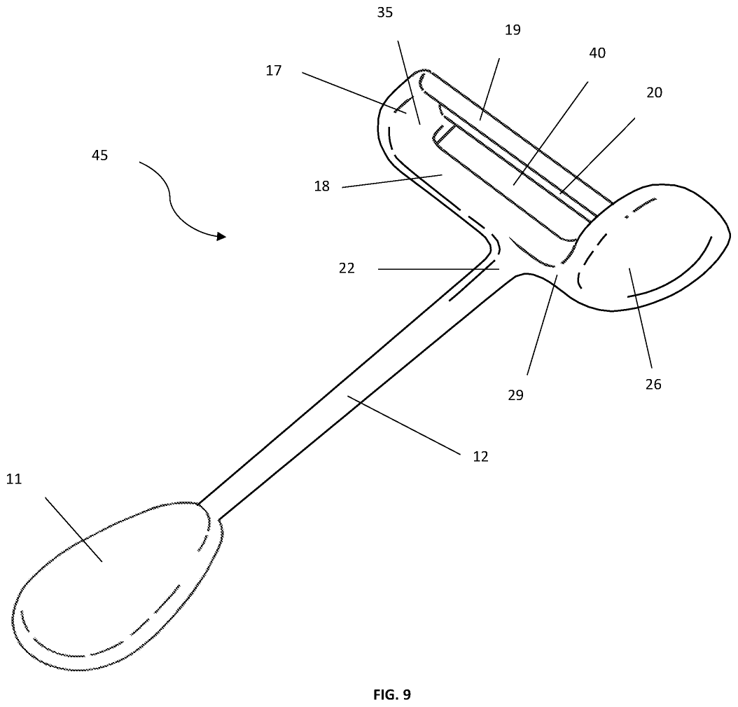

On the other hand, in FIGS. 6, 7, 8, and 9, it is shown the preferred embodiment 45 according to the invention. As embodiment 25, embodiment 45 also comprises handle section 11, connecting section 12 having its first end 21 connected to said handle section 11, a first holding section 14 which comprises first internal receiving channel 15 and second internal receiving channel 16, second holding section 17, supporting section 18, first inserting section 19 and second inserting section 20. However, as illustrated in FIGS. 6, 7, 8, and 9, in embodiment 45, the second end 22 of connecting section 12 is connected to the back side of the supporting section 18. The instant invention comprises any embodiment of the gauze pad holder wherein the said second end 22 of the connecting section 12 is connected to any position of the back side of the supporting section 18. The particular position of said connection as illustrated in FIGS. 6, 7, 8, and 9, should be understood as a preferred position, even though any potential changes of said position along the back side of supporting section 18 is within the scope of the instant invention.

More specifically, and as illustrated in any of FIGS. 2 to 9, the first holding section 14, comprises a main body comprising a smooth external side 26, a flat and smooth internal side 27, located in the opposite position to said external side 26; a front side 28, a back curved and smooth side 29. In some embodiments according to the invention, the external side 26 beside smooth is also convex. Said main body comprises rounded edges, suitable adaptable to the curvature of the oral cavity. It also comprises, a first internal receiving channel 15, having an opening or aperture 30, having exits or in direct communication with the flat and smooth internal side 27 and the front side 28 of said first holding section 14. As illustrated in, for example in any of the FIGS. 1, 2, 3, 6, 8, and 10, underneath said first internal receiving channel 15, the main body of first holding section 14 also comprises a second internal receiving channel 16, which has the same design and structure as the first internal receiving channel 15; thus, it has an exit or aperture 30a that is in direct communication with the flat internal side 27 and the front side 28 of said first holding section 14. It is pointed out, that none of the internal receiving channels 15 and 16 have any exit to the external side 26 of the main body of the first holding section 14. The particular design of internal receiving channel 15 comprises two circular carved-out sections, 39a and 39b with a narrow path in between. The same design is also found in the internal receiving channel 16, which comprises the two circulars carved-out sections 39c and 39d having a narrow path in between, as illustrated in FIGS. 2 and 6.

In regard to the supporting section 18, as shown in any of the FIGS. 1 through 9, it comprises a rectangular main body having a first end 31, which is connected to the first holding section 14; a second end 32, which is connected to the second holding section 17, a front side 33, and a back side 34.

All of the embodiments of the gauze pad holder according to the invention also comprises a second holding section 17, located in the opposite location of the first holding section 14, and connected to the second end 32 of the supporting section 18. Said second holding section 17 comprises a main body having an external side 23 with a smooth surface, a rounded back side 24, a flat and smooth internal side 35, and a front side 36. Embodiments comprising the external smooth surface 23 with a convex configuration are also within the scope of the instant invention.

From the upper part of the front side 36 of the second holding section 17, is connected to the first inserting section 19. It comprises a main extended cylindrical body, which comprises a disk-shaped first end 37 and a second angled end 38, which is connected to the upper section of the front side 36 of the second holding section 17 at its most outward area of the said angled section, thus forming the indentation 39, that allows the cylindrical body of the inserting section 19 to be suitably moved toward the upper section of the front side 28 of the first holding section 14. Said cylindrical body of first inserting section 19 may be flexible, semi-flexible, or inflexible.

Similarly, at the lower part of said front side 36 of the second holding section 17 and, thus, underneath the first inserting section 19, it is located second inserting unit 20; which is identical in structure and shape to the first inserting section 19 and thus, it comprises a cylindrical body, having a disk-shaped end 37a, second angled end 38a, which is connected to the lower part of the front side 36 of the second holding section 17, creating indentation 39a, that allows the cylindrical body of the second inserting section 20 to also be moved toward the front side 28 of the first holding section 14. Said cylindrical body of inserting section 20 may be flexible, semi-flexible, or inflexible.

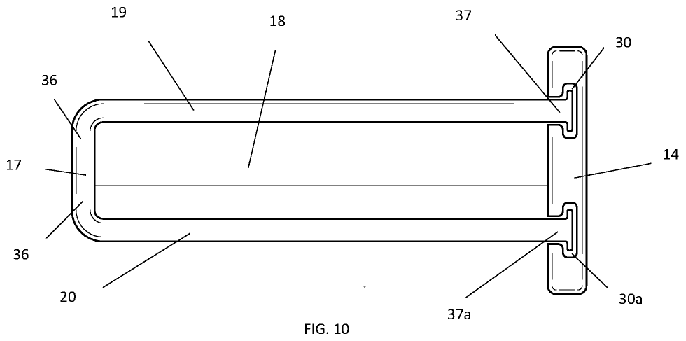

As illustrated, for instance in FIGS. 2, 3, 6, and 7, any embodiment of the gauze pad holder 10 has an open configuration whenever the first inserting section 19 and the second inserting section 20 have their disk-shaped ends 37 and 37a free or not connected to any other section of the holder 10. Alternatively, in FIGS. 1, 4, 5, 8, and 9, the closed configuration of the gauze pad holder 10 is achieved by moving the cylindrical bodies of the first and second inserting sections 19 and 20, respectively, toward the front side 28 of the first holding section 14, and further inserting the disk-shaped ends 37 and 37a of said inserting sections 19 and 20 into the apertures 30 and 30a, respectively, thus, into the interior of the first internal receiving channel 15 and into the interior of the second internal receiving channel 16, respectively, and in such a manner that internal cavity 40 is created once the disk-shaped ends 37 and 37a are in the interior of the first holding section 14 and thus inside the respective internal receiving channels 15 and 16. As illustrated in FIG. 10, the particular shape of the internal receiving sections 15 and 16 and thus, apertures 30 and 30a, respectively, are designed to properly receive and secure the corresponding disk-shaped ends 37 and 37a of the inserting sections 19 and 20, respectively. Carved-out circular sections 39a and 39b in the internal receiving channel 15 are particularly designed to received and to house the disk-shaped end 37 of inserting section 19; while carved-out sections 39c and 39d in the internal receiving channel 16 are particularly designed to received and a house the disk-shaped end 37a of the inserting section 20.

Thus, the particular design of the internal channels 15 and 16 allow the ends 37 and 37a of inserting sections 19 and 20 to be introduced, slid, and secured inside the corresponding receiving channels sections 15 or 16, preferably until end 37 is housed in the carved-out section 39b and end 37a is housed in carved out section 39d, as illustrated in, for example, FIGS. 4 and 8; wherein apertures 30 and 30a, respectively, are also shown at the internal flat side 27 of first holding section 14. The configuration of apertures 30 and 30a, as illustrated in FIG. 10 from the perspective of the front side 28 of the first holding section 14, allows the entering and insertion of the disk-shaped ends 37 and 37a, respectively.

As illustrated, in the close configuration of the gauze pad holder 10, disk-shaped ends 37 and 37a are enclosed and secured in a nonpermanent manner within the main body of first holding section 14, in this manner are not exposed outside of the said main body, avoiding that said ends 37 ad 37a are not in contact with the patient gums in any moment once the gauze pad holder 10 is in use.

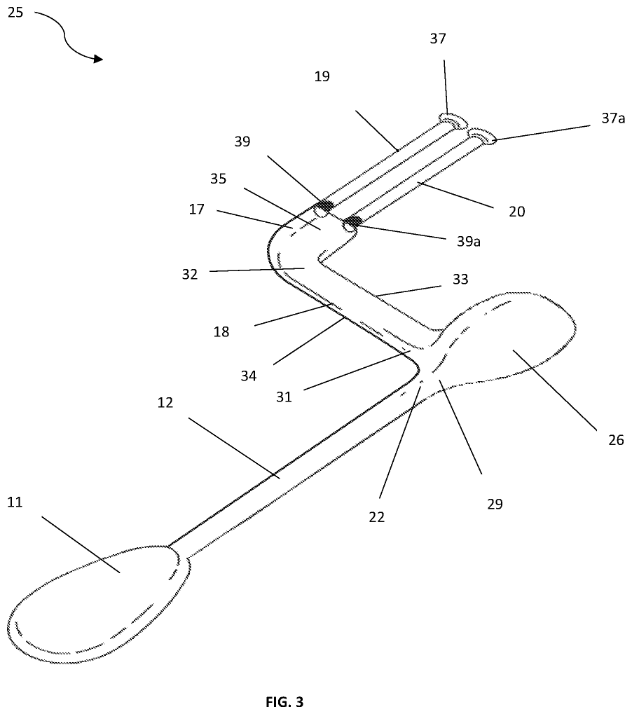

As indicated previously, FIG. 2 illustrates embodiment 25, showing the first holding section 14 positioned to the left and the second holding section 17 positioned to the right. The illustration of FIG. 3, results after the rotation of the holder as illustrated in FIG. 2, in order to illustrate the embodiment 25 having the second holding section 17 positioned to the left and the first holding section be positioned to the right. In both, FIGS. 2 and 3, embodiment 25 is illustrated with disk-shaped ends 37 and 37a free, thus the holder is illustrated in an open configuration. FIGS. 2 and 3 are presented in order to illustrate the different details of embodiment 25, such as the connectivity of the different parts, and their rounded edges. In FIGS. 4 and 5, the same embodiment 25 is shown in different positions and a closed configuration, having the disk-shaped end 37 and the disk-shaped end 37a already inserted into and confined to the interior of the internal receiving channels 15 and 16, respectively, and illustrating the internal cavity 40, suitable to receive and to secure a gauze pad 42.

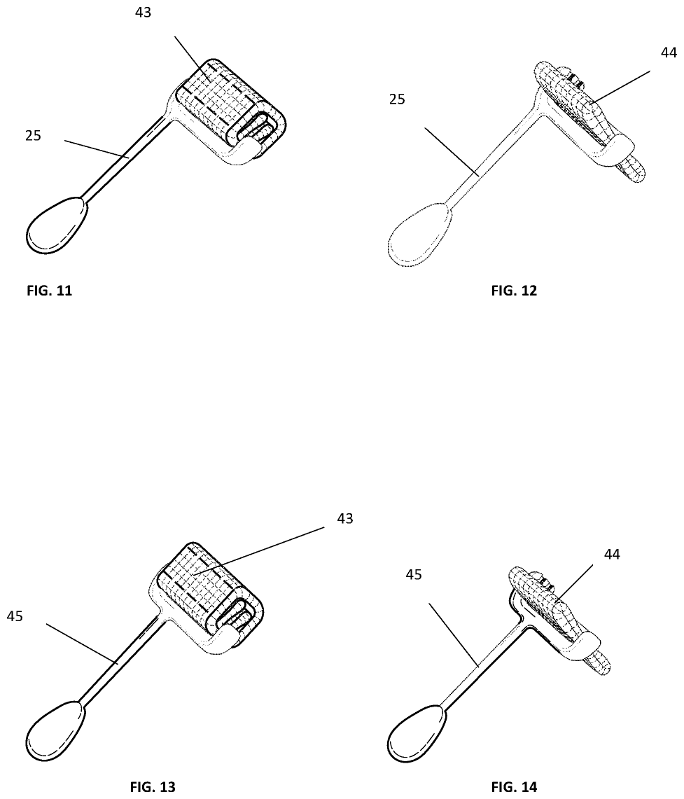

Gauze pad 42 may be assembled and firmly secured in the interior of said cavity 40. The gauze pad 42 may be assembled in different manners within the same embodiment of the holder 10 since all the essential parts of said embodiments are identical as previously discussed. In other words, the fact that the gauze pad may be assembled in different manners in the same type of embodiment of the holder 10, adds versatility to the use of said holder 10. For instance, FIG. 11, illustrates embodiment 25 having gauze pad 43, which is already assembled in a rolled-up manner; while in FIG. 12, the same embodiment 25 is presented with gauze pad 44 already which is assembled in a folded-up manner. Similarly, embodiment 45 is illustrated in FIG. 13 having gauze pad 43 assembled in a rolled-up manner while in FIG. 14 the same embodiment 45 is presented with gauze pad 44, which is assembled in a folded-up manner. The gauze pad 42 may be assembled in any other suitable manner, which depends on the particular shape or configuration of the postsurgical site requiring its use in order to properly cover and effectively press said postsurgical area.

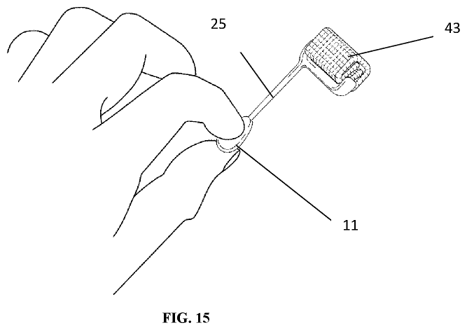

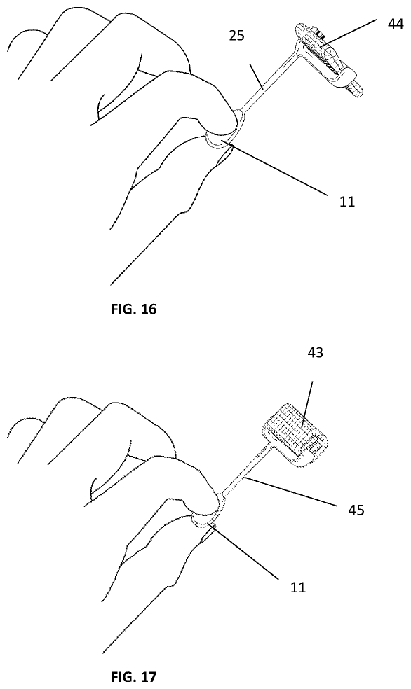

As illustrated in FIGS. 15 to 18, the suitable and proper manner to hygienically grab the embodiments of the gauze pad holder 10 with a gauze pad 42 already assembled and ready to use is by grabbing the handle 11 by its center, which is preferably concave, without the need to touch the gauze pad with the fingers or exposing it to any external surface.

All conceivable embodiments within the scope of the instant invention, including but not limited to preferred embodiments 25 and 45 may be made of any suitable strong material, such as plastic or foam. Preferably may be made of plastic, via suitable molding techniques known in the art. It is contemplated that the embodiments may have different sizes since the holder may be used in patients of different ages. It is contemplated that the herein disclosed holder may be for disposable use.

In operational terms, the gauze pad holder herein described is designed so that a cavity 40 is formed after inserting the disk-shaped ends 37 and 37a of the cylindrical bodies of inserting sections 19 and 20 into the internal receiving channels 15 and 16 on the main body of the first holding section 14, in order to provide cavity or chamber 40, wherein gauze pad 42, assembled in any suitable manner, may firmly be held and secured in a suitable assembling manner under hygienic conditions after disinfection. The gauze pad holder 10 is ergonomic and of a minimum nuisance to the patient since its external sides are convex and smooth, and easy to handle; thus, it avoids mayor discomforts to the patient. In this way, a properly assembled and disinfected gauze pad 42 is ready to use by a patient that, regularly does not how to properly assemble said gauze pad 42, since most of them are not experts in the oral surgery field. Delegating such tasks to the patients usually increases the risk of bleeding and other postoperative complications because of lack of experience or knowledge in properly assembling said gauze pad 42 or even further properly setting it on the postsurgical intraoral site.

As illustrated in FIGS. 15 to 18, the gauze pad holder herein disclosed may be manipulated utilizing its handle with no need of touching the gauze or exposing it to microorganisms or any other contaminants such as bacteria or virus. It further provides a hygienic alternative to avoid the need to introduce the patient's fingers inside the mouth, especially under post-surgical conditions, during the process of placing the gauze pad in the oral cavity. Furthermore, the secured gauze pad 42 already disinfected and assembled in the holder according to the invention also has an ergonomic shape or design with a smooth external surface, that is necessary to obtain a suitable direct contact of the gauze pad with the gums and alveolar socket after an intraoral surgery has been performed. Such direct contact is fundamental for a successful and safe healing process that is highly sought when providing high-quality treatment.

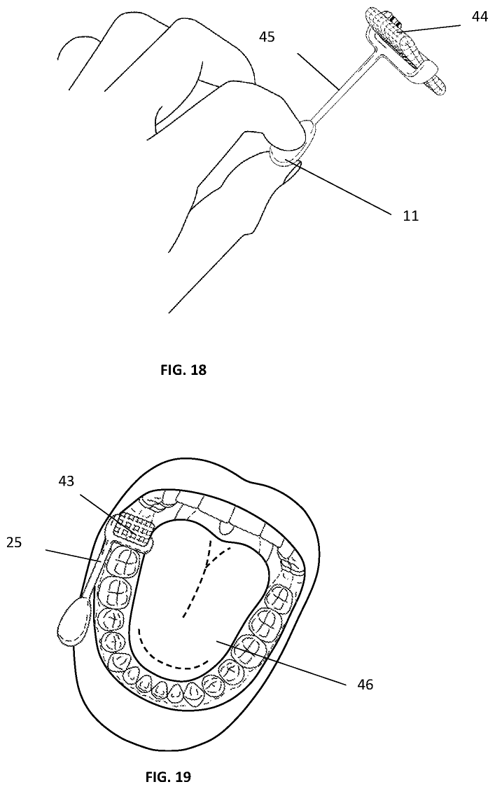

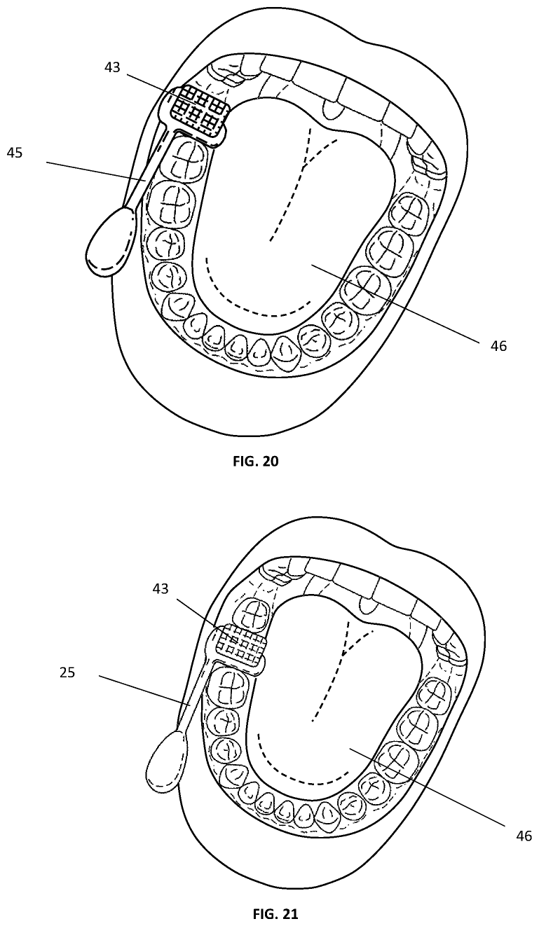

FIGS. 19 to 24, illustrate the placing of the gauze pad holder, according to the invention with an already assembled disinfected gauze pad in a patient's mouth 46. FIG. 19 shows a perspective view of the postsurgical use of the embodiment 25 of the gauze pad holder, as illustrated in FIG. 11, being used in the socket located at the third molar tooth site after the intraoral surgical procedure has been performed. FIG. 20 shows a perspective view of the postsurgical use of the embodiment 45 of the gauze pad holder as illustrated in FIG. 13, as used in the socket located at the third molar tooth site after the intraoral surgical procedure has been performed. Similarly, FIG. 21 shows a perspective view of the postsurgical use of the embodiment 25 of the gauze pad holder illustrated in FIG. 11, used in the socket located at the second molar tooth site after the intraoral surgical procedure has been performed, while in FIG. 22 it is shown a perspective view of the postsurgical use of the embodiment of the gauze pad holder illustrated in FIG. 14, used in the socket positioned at the first molar tooth site after the intraoral surgical procedure has been performed. On FIG. 23, it is illustrated a perspective view of the postsurgical use of the embodiment 25 of the gauze pad holder illustrated in FIG. 12, used in the socket at the canine tooth site after the intraoral surgical procedure has been performed and on FIG. 24 it is illustrated a perspective view of the postsurgical use of the embodiment 45 of the gauze pad holder illustrated in FIG. 14, used in the socket located at the central incisor tooth site after the intraoral surgical procedure has been performed. It should be understood that any embodiment of the gauze pad holder 10 comprising an already disinfected and firmly secured and assembled gauze pad 42 may be used in any socket after an intraoral surgical procedure or extraction of a tooth in the upper or lower jaw has been performed.

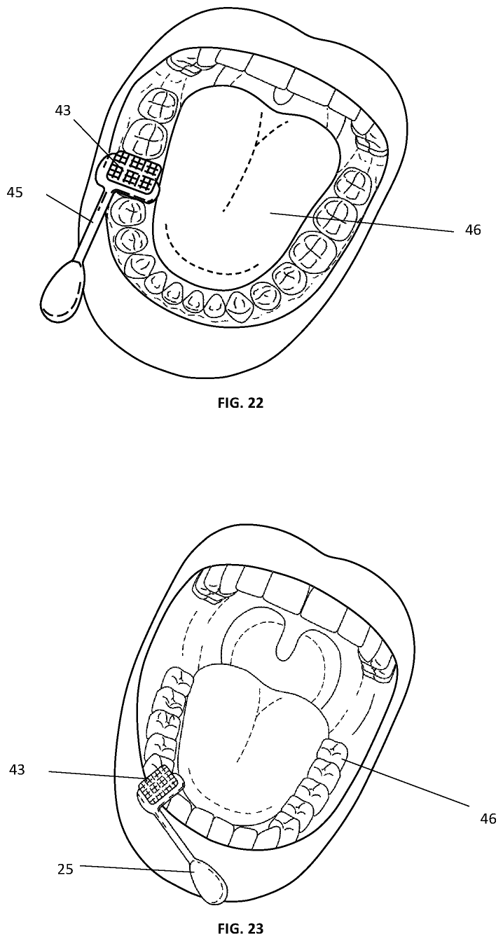

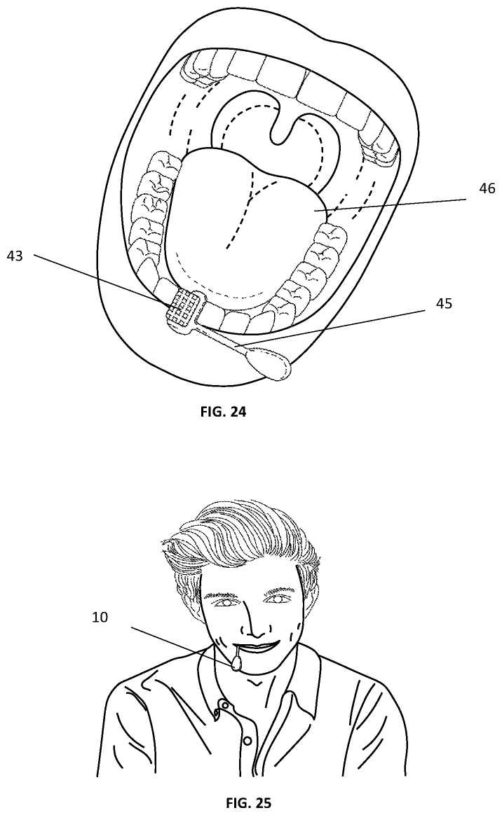

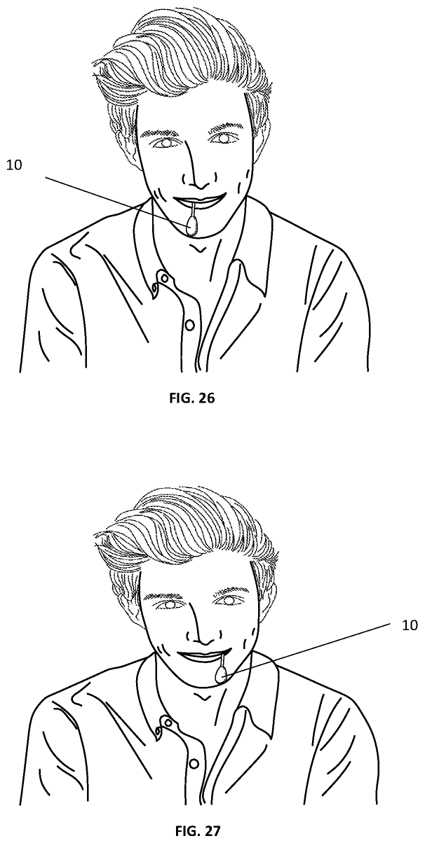

After the disinfected and firmly secured gauze pad has been assembled in the particular embodiment of the holder 10 is placed on the pertinent postsurgical intraoral area, and the patient presses the gauze pad 42 by closing the jaws as illustrated in FIGS. 24 to 26, said gauze pad 42 should be maintained in the surgical area for about 30 minutes in order to maintain the socket area dry of excess saliva and blood and to further allow the blood clot to properly form. As indicated previously, any embodiment of the herein disclosed holder is ergonomically designed according to the anatomy of the oral cavity and the surroundings of the given surgical site.

While the patient is using the herein described holder, the patient may secure and control said holder at any given time, if necessary, by holding the handle section; thus, the patient may even talk carefully without the gauze pad being displaced from its holder. If necessary, the gauze pad holder already having an assembled gauze pad 42 may be substituted by a new one by just repeating the described process. Additionally, any gauze pad holder according to the invention, and having a gauze pad 42 already assembled may be used as a support for antibiotic creams, anesthetic unguents, or antiseptic compositions used in the treatment of gum bites, wounds, or any other gum conditions requiring such medical compositions.

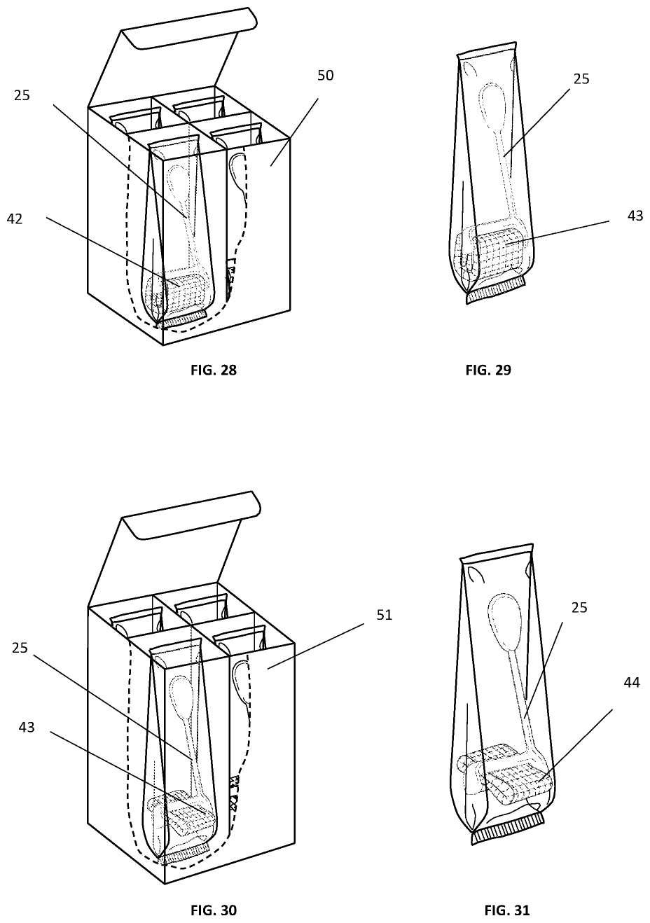

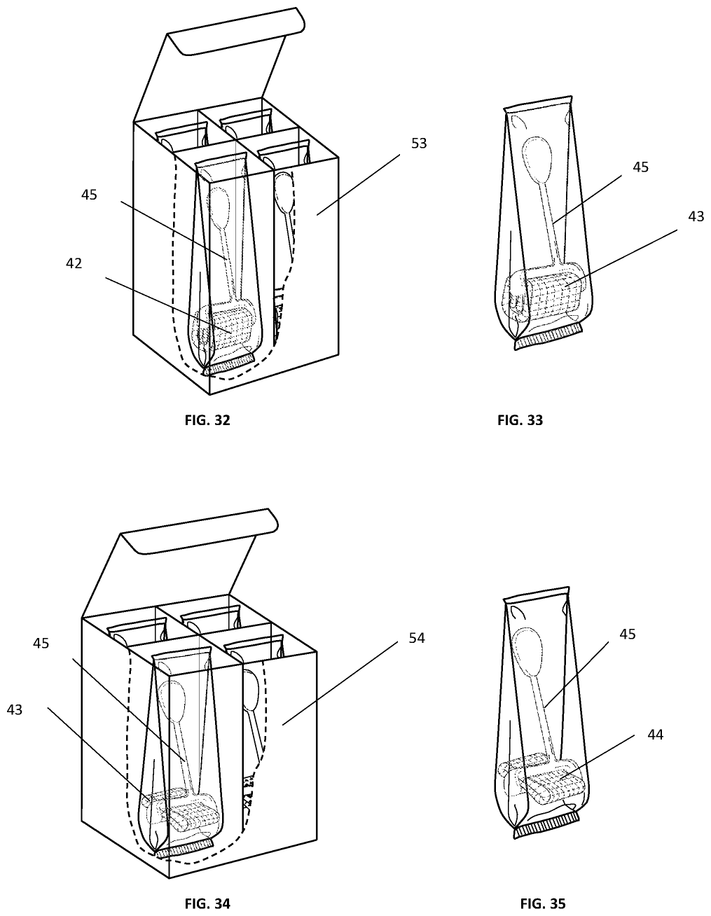

Any embodiment of the holder within the scope of the instant invention may be sold in enclosed, sanitary, and hygienic kits or packages, similar to packages 50 and 51 as illustrated in FIG. 28 to 31 for embodiment 25; or in similar to packages 53 and 54 as in FIG. 32 to 35 for embodiment 45. Each of said kits comprise multiple individually wrapped gauze pad holder 10, comprising a gauze pad 42, already assembled in said holder 10, and wherein said gauze pad 42 have been already and properly disinfected and sanitized by known methods in the art and wherein said gauze pad 42 may be assembled in the holder 10 in any suitable and convenient manner in order to avoid potential health risks caused by microorganisms such as germs and/or bacteria. Thus, the manner in which the gauze pad 42 is assembled in FIGS. 32 to 35 are only for illustration purposes and the scope of the invention includes packages containing any embodiment of the gauze pad holder 10 comprising said gauze pad 42 assembled in any other suitable and convenient manner.

Although the invention has been described and illustrated in detail, it is to be clearly understood that such description is for purposes of illustration and example, and it is not intended to be taken by way of limitation. For instance, some sections of the gauze holder such as the handle section, connecting section, the supporting section, and/or any of the holding sections may have alternatives shapes and/or configurations and still be within the spirit of the invention. Therefore, it is recognized that multiple variations exist, including both narrowing and broadening variations of the appended claims.

What is claimed is:

1. A holder, useful in holding a gauze pad in site after an intraoral surgical procedure, said holder comprising:
a) a handle section;
b) a connecting section, said connecting section comprising:
i. an elongate body having a first end and a second end;
c) a first holding section comprising a main body said main body comprising:
i. an external side;
ii. a flat internal side located in the opposite position to said external side;
iii. a front side;
iv. a back side;
v. rounded edges;
d) a first internal receiving channel comprising an aperture, which is in direct communication with said front side and with said flat internal side;
e) a second internal receiving channel, located underneath said first internal receiving channel, said second internal receiving channel comprising an aperture, which is in direct communication with the front side and with the flat internal side of said main body;
e) a supporting section comprising a rectangular main body, said main body comprising:
i. a first end;
ii. a second end;
iii. a front side;
iv. a back side;
f) a second holding section comprising a main body, said main body comprising:
i. an external side;
ii. a flat internal side;
iii. a back side;
iv. a front side;
g) a first inserting section comprising a cylindrical main body said extended cylindrical main body comprising:
i. a disk-shaped first end;
ii. a second angled end;
h) a second inserting section, comprising a cylindrical main body, said extended cylindrical main body comprising:
i. a disk-shaped first end;
ii. a second angled end;
wherein:
the first end of the elongate body of the connecting section is connected to said handle section;
the second end of the elongate body of the connecting section is alternatively connected (1) directly and in an aligned position to the first holding section, or (2) directly to the supporting section; or (3) directly and in aligned position to the second holding section;
the first end of the supporting section is perpendicularly connected to the internal flat side of the first holding section;
the second end of the supporting section is perpendicularly connected to the flat internal side of the second holding section;
the second angled end of the extended cylindrical main body of the first inserting section is connected to the front side of the second holding section; and
the second angled end of the extended cylindrical main body of the second inserting section is connected to the front side of the second holding section in a parallel position regarding the extended cylindrical main body of the first inserting section; and
wherein:
the described connections between the handle section (a), the connecting section (b), the first holding section (c), the supporting section (d), the second holding section (e), the first insertion (f), and the second insertion section (g) constitute and provide a holder having a single integral structure; and
wherein:
an internal cavity, capable of holding and secure a gauze pad is created by inserting the disk-shaped first end of the first inserting section into the first internal receiving channel of the first holding section via the aperture that is in direct communication with the front side and with the flat internal side of said first holding section and by inserting the disk-shaped first end of the second inserting section into the second internal receiving channel of the first holding section via the opening that is in direct communication with the front side and with the flat internal side of said first holding section.

2. The holder as recited in claim 1, further comprising a smooth and convex surface at the external sides of the first and second holding sections.

3. The holder as recited in claim 1, further comprising carved out circular sections inside the first and the second internal receiving channels.

4. The holder as recited in claim 1, wherein the second end of the connecting section is connected directly, and in an aligned position to the first holding section.

5. The holder as recited in claim 1, wherein the second end of the connecting section is connected directly, and in an aligned position to the second holding section.

6. The holder as recited in claim 1, wherein the second end of the connecting section is connected to the supporting section.

7. The holder as recited in claim 6, wherein the second end of the connecting section is connected to the back side of the supporting section.

8. The holder as recited in claim 4, wherein the second end of the connecting section is connected between the center of the back side of the supporting section and the first holding section.

9. The holder as recited in claim 4, wherein the handle section is flat.

10. The holder as recited in claim 8, wherein the handle section is flat.

11. The holder as recited in claim 4, wherein the handle section has a concave center.

12. The holder as recited in claim 8, wherein the handle section has a concave center.

13. The holder as recited in claim 4, wherein the elongate body of the connecting section is cylindrical.

14. The holder as recited in claim 8, wherein the elongate body of the connecting section is cylindrical.

15. The holder as recited in claim 4, wherein said holder is made of plastic.

16. The holder as recited in claim 8, wherein said holder is made of plastic.

17. The holder as recited in claim 4, further comprising a smooth and convex surface at the external sides of the first and second holding sections.

18. The holder as recited in claim 4, further comprising carved out circular sections inside the first and the second internal receiving channels.

19. The holder as recited in claim 7, further comprising a smooth and convex surface at the external sides of the first and second holding sections.

20. The holder as recited in claim 8, further comprising carved out circular sections inside the first and the second internal receiving channels.

21. A sanitary and disposable kit comprising a predetermined amount of individually wrapped units, each comprising:
i. a holder a recited in claim 1;
ii. a gauze pad already assembled and properly assembled and secured on the internal cavity of said holder; and
iii. an enclosure containing said predetermined amount of individually wrapped units.

22. A sanitary and disposable kit comprising a predetermined amount of individually wrapped units, each comprising:
i. a holder a recited in claim 4;
ii. a gauze pad already assembled and properly assembled and secured on the internal cavity of said holder; and
iii. an enclosure containing said predetermined amount of individually wrapped units.

23. A sanitary and disposable kit comprising a predetermined amount of individually wrapped units, each comprising:
i. a holder a recited in claim 8;
ii. a gauze pad already assembled and properly assembled and secured on the internal cavity of said holder; and
iii. an enclosure containing said predetermined amount of individually wrapped units.
